(19) United States
(12) Patent Application Publication
(10) Pub. No.: US 2006/0052633 A1
(43) Pub. Date: Mar. 9, 2006

(54) Method for preparing organic acid

(76) Inventors: Sang-gi Lee, Daejeon-si (KR); Ji-joong Moon, Daejeon-si (KR); Dae-sun Rew, Daejeon-si (KR); Dong-hyun Ko, Daejeon-si (KR); Sung-shik Eom, Daejeon-si (KR)

Correspondence Address: CANTOR COLBURN, LLP, 55 GRIFFIN ROAD SOUTH, BLOOMFIELD, CT, 06002, US

(21) Appl. No.: 10/516,901

(22) PCT Filed: Jun. 4, 2004
(86) PCT No.: PCT/KR04/01350

§ 371 Date: Dec. 3, 2004

(30) Foreign Application Priority Data
Jun. 5, 2003 (KR) 10 2003 0036352

(51) Int. Cl. C07C 51/235; C07C 51/16

(52) U.S. Cl. 562/531

(57) ABSTRACT

Provided is a method for producing an organic acid, which includes: mixing a compound containing one or two aldehyde groups and a solvent to obtain a reaction mixture; and maintaining the reaction mixture in a liquid phase in the presence of pure oxygen or O₂-enriched air containing 25-90% oxygen at a temperature of a 0-70° C., under a pressure condition of an atmospheric pressure to 10 kg/cm², and for 2-10 hours. The method can enhance the selectivity of the organic acid, thereby increasing the yield of the organic acid.

### TECHNICAL FIELD

The present invention relates to a method for producing an organic acid. More particularly, the present invention relates to a method for producing an organic acid, which includes mixing a hydrocarbon containing one or more aldehyde groups and a solvent and maintaining the reaction mixture in a liquid phase in the presence of pure oxygen or O₂-enriched air containing at least 50% oxygen.

### BACKGROUND ART

Generally, production of an organic acid by liquid-phase oxidation of aldehyde is well known. Oxidation of aldehyde is carried out with oxygen or air in the presence or absence of a catalyst. Even though gas-phase oxidation of aldehyde is possible, an organic acid is generally produced by liquid-phase oxidation of aldehyde in the absence of a solvent.

In both catalytic and non-catalytic oxidation of aldehyde, a percarboxylic acid is produced as a reaction intermediate. Oxidation of aldehyde is mainly carried out in a reactor made of stainless steel. A reactor coated with glass or enamel can also be used.

In catalytic oxidation of aldehyde, a metal salt is mainly used as a catalyst. Generally, it is known that a noble metal salt or a transition metal salt having one or more acid numbers is mainly used as a catalyst. However, since a catalyst component may cause problems associated with environmental contamination, separation and recovery of the catalyst component after the oxidation are required. For this reason, in recent years, there is a tendency of gradual increase in use of non-catalytic oxidation of aldehyde.

On the other hand, in non-catalytic oxidation of aldehyde, to more efficiently carry out the oxidation of aldehyde with oxygen, it is more important to increase the solubility of the oxygen by completely dispersing the oxygen in a reaction solution. Generally, when the conversion rate of the aldehyde reaches 90-95% in a single reactor, a reaction rate decreases. In view of this problem, unreacted aldehyde can be reused after distillation recovery of a reaction product or sequential oxidation using additional reactors can be carried out. By doing so, it is known that the conversion rate of aldehyde can reach 99% or more. However, since a boiling point difference between the aldehyde, which is a raw material, and an ester compound (although its content is only several %), which is a byproduct of the oxidation of the aldehyde, is small, distillation separation of the ester compound is difficult, thereby lowering the selectivity of a reaction product. For example, selectivity of an organic acid by oxidation of aldehyde may be 93-94% for an aldehyde compound with 4-6 carbon atoms but only 85% for an aldehyde compound with 7 or more carbon atoms. In this regard, improvement of the yield of an organic acid by increasing the selectivity of the organic acid is required. Hitherto, there are several patent documents about enhancements of the conversion rate of aldehyde, including Japanese Patent Laid-Open Publication Nos. 53-108915, 53-13223, 53-13225, and 55-17131, U.S. Pat. No. 4,350,829, and European Patent No. EP1073621. However, no patent documents about enhancements of the selectivity of a reaction product are reported.

### DISCLOSURE OF THE INVENTION

In view of these problems, the present invention provides a method for producing ah organic acid by mixing an aldehyde compound with a solvent followed by liquid-phase oxidation. According to the method, since the organic acid can be easily separated from the aldehyde compound used as a raw material and solvents with good miscibility are used in a reaction system, a higher purity organic acid can be produced in a higher yield, as compared to a conventional technique.

The above and other objects of the present invention can be accomplished by embodiments of the present invention as will be described hereinafter.

According to an aspect of the present invention, there is provided a method for producing an organic acid, which includes: mixing a compound containing one or two aldehyde groups and a solvent to obtain a reaction mixture; and maintaining the reaction mixture in a liquid phase in the presence of pure oxygen or O₂-enriched air containing 25-90% oxygen at a temperature of 0-70° C., under a pressure condition of an atmospheric pressure to 10 kg/cm², and for 2-10 hours.

The solvent may be used in an amount of 1-55 wt %, based on 100 wt % of the aldehyde group-containing compound.

The aldehyde group-containing compound may be selected from the group consisting of formaldehyde, acetaldehyde, propionaldehyde, n-butyraldehyde, i-butyraldehyde, 2-methylbutyraldehyde, n-valeraldehyde, caproaldehyde, heptylaldehyde, nonylaldehyde, and 2-ethylhexylaldehyde.

The solvent may be selected from the group consisting of ketones, alcohols, esters, ethers, hydroxyl group-containing compounds, and a mixture thereof.

According to another aspect of the present invention, there is provided an organic acid produced by the method.

Hereinafter, the present invention will be described in detail.

In the present invention, the aldehyde group-containing compound (hereinafter, also called as “aldehyde compound”) used as a raw material can be generally prepared by hydroformylation. The purity of the aldehyde compound does not significantly affect reactivity but is preferably about 90% or more and more preferably 95% or more. As used herein, the term “aldehyde group-containing compound” refers to a straight or branched alkyl group of a R—CHO structure where R is H or 2-8 carbon atoms. Representative examples of the aldehyde group-containing compound include formaldehyde, acetaldehyde, propionaldehyde, n-butyraldehyde, i-butyraldehyde, 2-methylbutyraldehyde, n-valeraldehyde, caproaldehyde, heptylaldehyde, and nonylaldehyde. In addition, examples of the aldehyde group-containing compound include phenylacetylaldehyde, benzaldehyde, o-tolualdehyde, m-tolualdehyde, p-tolualdehyde, salycylaldehyde, p-hydroxybenzaldehyde, anisaldehyde, vanilin, piperonal, 2-ethylhexylaldehyde, 2-propylheptylaldehyde, 2-phenylpropionaldehyde, 2-[p-isophenyl]propionaldehyde, and 2-[6-methoxy-2-naphtyl]propionaldehyde.

An oxygen molecule-containing gas used in the oxidation of the aldehyde compound is pure oxygen or oxygen diluted with an inert gas such as nitrogen, helium, argon, or carbon dioxide. Generally, in the presence of the oxygen molecule-containing gas, 99% or more of the aldehyde compound is converted to an organic acid in one or more continuous or batch reactors.

Generally, production of organic acids by oxidation of aldehyde compounds is accomplished by consecutive reaction of carbon radicals produced by dissociation of hydrogen atoms from aldehyde groups, with oxygen and aldehyde groups. At this time, some byproducts may be produced by dissociation or side reaction of the carbon radicals. The content of the byproducts slightly varies according to the types of the aldehyde compounds. Generally, the content of the byproducts is 4-6% for an aldehyde compound with 4-6 carbon atoms, which provides 90-94% yield of an organic acid. On the other hand, the content of the byproducts is 12-15% for an aldehyde compound with 7 or more carbon atoms, which provides up to 85% yield of an organic acid. Furthermore, since it is difficult to separate these byproducts from aldehyde compounds used as a raw material, the byproducts are used as a fuel oil or classified as a waste oil, thereby causing an economical loss. In this regard, it can be seen that prevention of such byproducts is essential for high yield of organic acids.

In the present invention, the solvent used in the oxidation of the aldehyde compound is preferably a hydrocarbon compound satisfying the following requirements: a) non-reactivity with pure oxygen or air containing 50% or more oxygen; b) inclusion of an oxygen atom or molecule on a hydrocarbon ring or end portion; c) partial or complete mixing with the aldehyde compound; and d) easy separation and purification from the aldehyde compound and the organic acid after the oxidation. Representative examples of such a hydrocarbon compound include ketones (e.g., acetone), alcohols (e.g., methanol), esters (e.g., ethylacetate), and ethers (e.g., dimethylether). The hydrocarbon compound may also be a hydroxyl group-containing compound such as monoethanolamine and ethyleneglycol. The above-described hydrocarbon solvents may be used alone or in combination. However, the present invention is not limited to the above-described solvents. Since the content of the solvent directly affects the selectivity of the organic acid, it must be determined by several experiments. Generally, the solvent is used in an amount of 1-55 wt %, preferably 5-50 wt %, based on 100 wt % of the aldehyde compound.

The oxidation of the aldehyde compound is carried out as follows.

The aldehyde compound and 1-55 wt % of one or more of the above-described solvents (based on 100 wt % of the aldehyde compound) are added in a reactor. Then, an inert gas such as nitrogen, helium, argon, or carbon dioxide is allowed to sufficiently flow in a reaction system and the reactor is set to a desired temperature. When the temperature of the reactor is maintained constant, pure oxygen or oxygen diluted with the above-described inert gas is added to the reactor to initiate the oxidation of the aldehyde compound.

A reaction temperature is in a range of 0-70° C. and preferably 5-60° C. If the reaction temperature is low, the selectivity of the organic acid may increase but an oxygen density in the reaction system may increase, thereby lowering stability. Therefore, oxidation at an excessively low temperature is not preferable. The oxidation may be carried out at an atmospheric pressure. However, the oxidation at a slightly pressurized condition can increase the solubility of oxygen, thereby ensuring a high conversion rate. At the same time, the selectivity of the organic acid can increase. In this regard, a reaction pressure may be in an range from an atmospheric pressure to 10 kg/cm² (gauge), and preferably 3-8 kg/cm² (gauge). Since the oxidation reaction produces a large quantity of heat, sufficient removal of the heat is required. If the heat of the oxidation reaction is not sufficiently removed, explosion may occur. A reaction rate is determined by a flow rate of oxygen and a heat removal method. A reaction duration is generally 2-10 hours and preferably 3-8 hours.

The organic acid that can be produced by the method of the present invention may be a carboxyl group-containing compound such as formic acid, acetic acid, propionic acid, butyric acid, valeric acid, caproic acid, capryic acid, capric acid, lauric acid, phenylacetic acid, benzoic acid, phthalic acid, isophthalic acid, terephthalic acid; adipic acid, 2-ethylhexanoic acid, isobutyric acid, 2-methylbutyric acid, 2-propylheptanoic acid, 2-phenylpropionic acid, 2-(p-isobutylphenyl)propionic acid, and 2-(6-methoxy-2-naphthyl)propionic acid.

In summary, the present invention provides a method for producing an organic acid in high yield by liquid-phase oxidation. More particularly, the present invention provides a method for producing a high purity organic acid in high yield by liquid-phase oxidation of a hydrocarbon containing one or more aldehyde groups in a solvent in the presence of pure oxygen or O₂-enriched air containing at least 50% oxygen followed by purification. Use of an appropriate reaction temperature and removal of the heat of the oxidation reaction are most important in enhancement of the yield of the organic acid. According to the method of the present invention, the aldehyde compound which is a raw material and the organic acid which is a reaction product can be easily separated and solvents with good miscibility are used in an amount of 5-50 wt %. Therefore, the yield of the organic acid can be enhanced by 8-10%, as compared to that of a conventional technique.

### BEST MODE FOR CARRYING OUT THE INVENTION

Hereinafter, the present invention will be described more specifically by Examples. However, the following Examples are provided only for illustrations and thus the present invention is not limited to or by them.

### EXAMPLE 1

300 g of isobutylaldehyde and 30 g of water were added in a glass reactor with capacity of 1 liter. Nitrogen was allowed to sufficiently flow in the reactor and a reaction temperature was set to 5° C. When the temperature of the reactor was maintained constant, oxygen was gradually fed at a flow rate of 180 ml/min with stirring. As reaction proceeded, a reaction pressure gradually increased. When a final pressure reached 6 kg/cm² (gauge), the reaction was terminated. After the reaction termination, a product was analyzed using a non-limiting analytic system.

### EXAMPLE 2

The same reaction as in Example 1 was performed except that 50 g of 2-ethylhexylalcohol was used instead of water.

### EXAMPLE 3

The same reaction as in Example 1 was performed except that 300 g of 2-ethylhexylaldehyde was used instead of isobutylaldehyde.

### EXAMPLE 4

The same reaction as in Example 1 was performed except that 300 g of 2-ethylhexylaldehyde was used instead of isobutylaldehyde and a mixture of 25 g of ethanol and 25 g of 2-ethylhexylalcohol was used instead of water.

### EXAMPLE 5

The same reaction as in Example 1 was performed except that 300 g of 2-ethylhexylaldehyde was used instead of isobutylaldehyde and 30 g of methanol was used instead of water.

### EXAMPLE 6

The same reaction as in Example 5 was performed except that 225 g of isobutanol was used instead of methanol.

### EXAMPLE 7

The same reaction as in Example 5 was performed except that 95 g of methanol was used.

### EXAMPLE 8

The same reaction as in Example 1 was performed except that 300 g of propionaldehyde was used instead of isobutylaldehyde and 75 g of isopropylalcohol was used instead of water.

### EXAMPLE 9

The same reaction as in Example 1 was performed except that 300 g of valeraldehyde was used instead of isobutylaldehyde and 90 g of ethanol was used instead of water.

### COMPARATIVE EXAMPLE 1

300 g of isobutylaldehyde was added to a glass reactor with capacity of 1 liter and the reactor was set to a temperature of 25° C. When the temperature of the reactor was maintained constant, oxygen was gradually fed at a flow rate of 180 ml/min with stirring. As reaction proceeded, a reaction pressure gradually increased. When a final pressure reached 6 kg/cm² (gauge), the reaction was terminated and a product was analyzed.

### COMPARATIVE EXAMPLE 2

The same reaction as in Comparative Example 1 was performed except that 300 g of 2-ethylhexylaldehyde was used.

### COMPARATIVE EXAMPLE 3

The same reaction as in Comparative Example 1 was performed except that air containing 21% oxygen was used instead of oxygen.

The results of Examples and Comparative Examples are summarized in Table 1 below.

TABLE 1

| | | | | Organic acid |
|---|---|---|---|---|
| | | | Aldehyde conversion | selectivity |
| Example | Aldehyde compound | Solvent | rate (%) | (%) |
| Example 1 | Isobutylaldehyde 300 g | Water 30 g | 99.7 | 94.6 |
| Example 2 | Isobutylaldehyde 300 g | 2-ethylhexylalcohol 50 g | 99.5 | 97.5 |
| Example 3 | 2-ethylhexylaldehyde 300 g | 2-ethylhexylalcohol 50 g | 99.6 | 93.8 |
| Example 4 | 2-ethylhexylaldehyde 300 g | Ethanol 25 g + | 99.5 | 95.2 |
| | | 2-ethylhexylalcohol 25 g | | |
| Example 5 | 2-ethylhexylaldehyde 300 g | Methanol 15 g | 99.9 | 93.2 |
| Example 6 | 2-ethylhexylaldehyde 300 g | Isobutanol 225 g | 98.1 | 93.9 |
| Example 7 | 2-ethylhexylaldehyde 300 g | Methanol 95 g | 99.8 | 96.2 |
| Example 8 | Propionaldehyde 300 g | Isopropylalcohol 75 g | 99.3 | 93.1 |
| Example 9 | Valeraldehyde 300 g | Ethanol 90 g | 99.7 | 94.3 |
| Comparative | Isobutylaldehyde 300 g | — | 99.8 | 92.1 |
| Example 1 | | | | |
| Comparative | 2-ethylhexylaldehyde 300 g | — | 99.4 | 84.6 |
| Example 2 | | | | |
| Comparative | 2-ethylhexylaldehyde 300 g | — | 76.2 | 91.5 |
| Example 3 | | | | |

From Table 1, it can be seen that the method of the present invention provides a higher yield of an organic acid, as compared to a conventional method.

### INDUSTRIAL APPLICABILITY

As apparent from the above description, in a method of producing an organic acid from an aldehyde compound according to the present invention, a use of an appropriate solvent enhances the selectivity of the organic acid, as compared to a conventional technique, thereby increasing the yield of the organic acid. The organic acid produced by the method of the present invention can be efficiently used as a material for compounds such as a plasticizer, a solvent, a medical intermediate, or the like.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method for producing an organic acid, which comprises:
mixing a compound containing one or two aldehyde groups and a solvent to obtain a reaction mixture; and
maintaining the reaction mixture in a liquid phase in the presence of pure oxygen or O₂-enriched air containing 25-90% oxygen at a temperature of 0-70° C., under a pressure condition of an atmospheric pressure to 10 kg/cm², and for 2-10 hours.

2. The method of claim 1, wherein the solvent is used in an amount of 1-55 wt %, based on 100 wt % of the aldehyde group-containing compound.

3. The method of claim 1, wherein the aldehyde group-containing compound is selected from the group consisting of formaldehyde, acetaldehyde, propionaldehyde, n-butyraldehyde, i-butyraldehyde, 2-methylbutyraldehyde, n-valeraldehyde, caproaldehyde, heptylaldehyde, nonylaldehyde, and 2-ethylhexylaldehyde.

4. The method of claim 1, wherein the solvent is selected from the group consisting of ketones, alcohols, esters, ethers, hydroxyl group-containing compounds, and a mixture thereof.

5. An organic acid produced by the method of claim 1.
